(19) United States
(12) Patent Application Publication
(10) Pub. No.: US 2018/0028795 A1
(43) Pub. Date: Feb. 1, 2018

(54) DISSOLVABLE DILATOR

(71) Applicant: Mayo Foundation for Medical Education and Research, Rochester, MN (US)

(72) Inventors: Ryan C. Hutchinson, Jacksonville, FL (US); Todd C. Igel, Ponte Vedra Beach, FL (US)

(73) Assignee: Mayo Foundation for Medical Education and Research, Rochester, MN (US)

(21) Appl. No.: 15/546,127

(22) PCT Filed: Jan. 21, 2016
(86) PCT No.: PCT/US16/14305

§ 371 Date: Jul. 25, 2017

Related U.S. Application Data
(60) Provisional application No. 62/108,255, filed on Jan. 27, 2015.

(51) Int. Cl. A61M 29/02; A61M 25/00

(52) U.S. Cl. CPC A61M 29/02; A61M 25/0009; A61M 2210/1078; A61B 2017/00004

(57) ABSTRACT

Devices and methods can be used for the dilation of body conduits. For example, this document provides devices and methods for dilating a ureter using a dissolvable dilator element that is compatible with an ureteroscope system. Such a dissolvable dilator can be advanced into a patient's ureter via the urologic guidewire in front of the ureteroscope, giving immediate, one-step Cureteral access and offering less ureteral trauma at an economical price point.

### CROSS REFERENCE TO RELATED APPLICATIONS

This application claims the benefit of U.S. Provisional Application No. 61/108,255, filed Jan. 27, 2015. The disclosure of the prior application is considered part of and is incorporated by reference in the disclosure of this application.

### BACKGROUND

### 1. Technical Field

This document relates to devices and methods for the dilation of human body conduits. For example, this document relates to devices and methods for dilating a ureter using a dissolvable dilator element that is compatible with an endoscope or ureteroscope system.

### 2. Background Information

Many diseases are diagnosed or treated through the use of visualizing instruments within the human body. A frequent problem with the use of endoscopes is the encountering of a narrowed portion, either normal or pathologic, of the target organ. Various techniques exist for dilating or passing these narrowings. In Urology, kidney stones (renal lithiasis) are small, hard deposits that form inside kidneys. The stones are made of mineral and acid salts. Kidney stones have many causes and can affect any part of a urinary tract—from the kidneys to the bladder. Stones form when urine becomes concentrated, allowing minerals to precipitate in the urine. Ureteroscopy is increasingly becoming the preferred modality for treating kidney stones of nearly all locations and sizes. One critical step in performing ureteroscopy is accessing the ureter with the scope. This generally requires placement of a wire up the ureter and dilation of the lower portion of the ureter where it enters the bladder, either with a long rigid dilator or an inflatable dilating balloon. These represent high force methods of dilation and, in general, dilation carries patient risk proportional to the amount of force required to dilate. This adds a step, is traumatic to the ureter and incurs significant cost for the procedure.

Despite advancements in scope technology making the devices smaller than ever, ureteral dilation is frequently required and the dilators used today are generally the same traumatic dilators used in the early stages of ureteroscopy. Moreover, they require an additional step, dilating the ureteral orifice then withdrawing the ureteral dilator and attempting to pass the scope, which can be unsuccessful. Finally, because of their size and complexity, existing dilators are expensive, adding cost to an already costly case that uses multiple disposable products.

### SUMMARY

This document provides devices and methods for the dilation of body conduits. For example, this document provides devices and methods for dilating a ureter using a dissolvable dilator element that is compatible with an endoscopy or ureteroscope system. Such a dissolvable dilator can be advanced into a patient's ureter via the urologic guidewire in front of the ureteroscope, giving immediate, one-step ureteral access and offering less ureteral trauma at an economical price point.

In one implementation, a dilator device includes a member defining a tapered outer profile such that a first outer diameter of a first end portion of the member is smaller than a second outer diameter of a second end portion of the member. The member defines an inner lumen extending between the first end portion and the second end portion. The lumen is configured to receive a guidewire therein. The member is comprised of a material that is dissolvable within a human body without incurring harm to the human body.

Such a dilator device may optionally include one or more of the following features. The tapered outer profile may comprise a frustoconical, pseudopyramidal or otherwise tapered geometric shape. The first end portion may comprise a blunt or tapered end that is configured to be atraumatic to the human body. The material may essentially dissolve within the human body after being within the human body less than 10 minutes. The material may comprise a sugar-based material.

In another implementation, a dilator system includes a guidewire and a dilator device. The dilator device comprises a member defining a tapered outer profile such that a first outer diameter of a first end portion of the member is smaller than a second outer diameter of a second end portion of the member. The member defines an inner lumen extending between the first end portion and the second end portion. The lumen is configured to receive the guidewire therein. The member is comprised of a material that is dissolvable within a human body without incurring harm to the human body.

Such a dilator system may optionally include one or more of the following features. The system may further comprise an endoscope. The guidewire may be a urologic guidewire. The system may further comprise a ureteroscope. The material may essentially dissolve within the human body after being within the human body less than 10 minutes. The material may comprise a sugar-based material.

In another implementation, a method of performing a ureteroscopy procedure to a human patient includes advancing a urologic guidewire into the patient such that at least a distal end portion of the guidewire is positioned within a ureter of the patient; advancing, over the urologic guidewire, a dilator device; advancing, over the urologic guidewire and distally to the dilator device, a ureteroscope; dilating, by the dilator device, an orifice of the ureter; after the dilating, allowing the dilator device to dissolve in the patient; and after the dilating, advancing a distal end portion of the ureteroscope into the ureter. The dilator device may comprise a member defining a tapered outer profile such that a distal outer diameter of a distal end portion of the member is smaller than a proximal outer diameter of a proximal end portion of the member. The member may define an inner lumen extending between the proximal end portion and the distal end portion. The lumen receiving the guidewire therein. The member may be comprised of a material that is dissolvable within the patient without incurring harm to the patient.

Such a method of performing a ureteroscopy procedure to a human patient may optionally include one or more of the following features. The dilator device may dissolve in less than 10 minutes in the patient. The material of the dilator device may comprise a sugar-based material. The method may further comprise capturing, using the ureteroscope, at least a portion of a kidney stone from within the ureter.

Particular embodiments of the subject matter described in this document can be implemented to realize one or more of the following advantages. In some embodiments, the dissolvable dilators provided herein allow for dilation of body conduits and entry thereto in a single step process, allowing for faster access, less risk for patient injury, and decreased procedure times. In some embodiments, the dissolvable dilators provided herein are less traumatic to patients in comparison to conventional dilator devices, potentially resulting in less tissue trauma and better visualization. Additionally, in some embodiments the dissolvable dilators provided herein may be less expensive to purchase and use in comparison to conventional devices and methods.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of patient undergoing ureteroscopy procedure to remove a kidney stone in the patient's ureter.

FIG. 2 shows an example ureteroscope that is equipped with a compatible dissolvable dilator, in accordance with some embodiments provided herein.

FIG. 3 shows a distal end portion of an example ureteroscope that is equipped with a compatible dissolvable dilator, in accordance with some embodiments provided herein. In this view, the dissolvable dilator is separated from the distal tip of the ureteroscope so that the dissolvable dilator is clearly distinguishable.

FIG. 4 shows a distal end portion of an example ureteroscope that is equipped with a compatible dissolvable dilator, in accordance with some embodiments provided herein. In this view, the dissolvable dilator is in an operative position in relation to the distal tip of the ureteroscope.

FIG. 5 shows an example dissolvable dilator, in accordance with some embodiments provided herein.

Like reference numbers represent corresponding parts throughout.

### DETAILED DESCRIPTION

This document provides devices and methods for the dilation of human body conduits. For example, this document provides devices and methods for dilating a ureter using a dissolvable dilator element that is compatible with an endoscopy or ureteroscope system. Such a dissolvable dilator can be advanced into a patient's ureter via the urologic guidewire in front of the ureteroscope, giving immediate, one-step ureteral access and offering less ureteral trauma at an economical price point.

The devices and methods provided herein may also be used to treat other conditions, such as ureteral, urethral and urinary conduit strictures, both benign and malignant, congenital narrowings of the ureter, ureteral, urethral and renal pelvis tumors, or narrowings of other hollow viscus and tissues within fields utilizing endoscopy, including but not limited to Gastroeterology, Orthopaedics, General surgery and others.

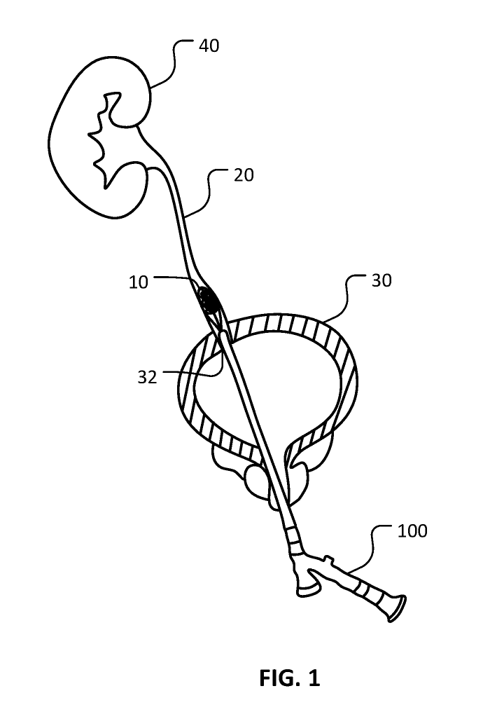

Referring to FIG. 1, a kidney stone 10 in a patient's ureter 20 can be removed using an ureteroscope 100 by performing a ureteroscopy procedure. Ureter 20 extends between the patient's bladder 30 and kidney 40. In this schematic depiction, only a single ureter 20 and kidney 40 are represented.

To access ureter 20 where kidney stone 10 resides, the distal tip portion of ureteroscope 100 must enter ureter 20 through a ureteral orifice 32 in the wall of bladder 30. This conventionally requires placement of a wire up ureter 20, and dilation of the lower portion of ureter 20 where it conflates with bladder 30 (i.e., the ureter ostium 32). Such dilation is conventionally performed either with a long rigid dilator or an inflatable dilating balloon. Using the conventional techniques, after dilation the dilator device is withdrawn, and then ureteroscope 100 can be advanced into ureter 20. Hence, one skilled in the art will appreciate that the conventional techniques of ureter dilation adds a procedural step, can be traumatic to ureter 20, and incurs significant cost for the ureteroscopy procedure. Moreover, because of their size and complexity, existing dilators are relatively expensive, adding cost to an already costly procedure that uses multiple disposable products.

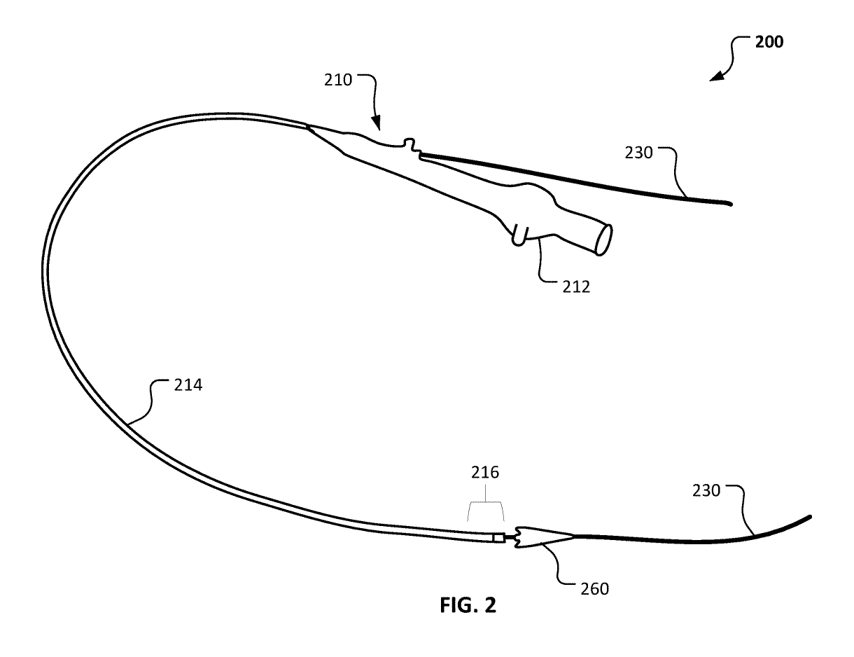

Referring to FIG. 2, in some embodiments an example compatible ureteroscope and dilator system 200 includes a ureteroscope 210, a urologic guidewire 230, and a dissolvable dilator 260.

Ureteroscope 210 can be any form of conventional ureteroscope. As such, ureteroscope 210 can include a proximal control handle 212, an elongate flexible or semi-rigid shaft 214, and a distal end portion 216. Elongate flexible or semi-rigid shaft 214 includes at least one channel (lumen) therethrough.

Urologic guidewire 230 is slidably coupled within the at least one channel of ureteroscope 210 from handle 212 to distal end portion 216. As such, elongate flexible shaft 214 can be installed over urologic guidewire 230 after urologic guidewire 230 has been placed within the patient. For example, for a ureteroscopy procedure at least a distal end portion of urologic guidewire 230 will be advanced to be positioned within the ureter of the patient.

Dissolvable dilator 260 is slidably disposed on urologic guidewire 230, and located distally of distal end portion 216. Accordingly, after placement of urologic guidewire 230, dissolvable dilator 260 serves as a leading-end member for elongate flexible shaft 214. Said another way, dissolvable dilator 260 precedes distal end portion 216 of shaft 214 as ureteroscope 210 is advanced farther into the patient. Because of the tapered profile of dissolvable dilator 260, dilator system 200 can be advanced into the patient, including the patient's ureter, with less trauma in comparison to ureteroscope 210 without dissolvable dilator 260.

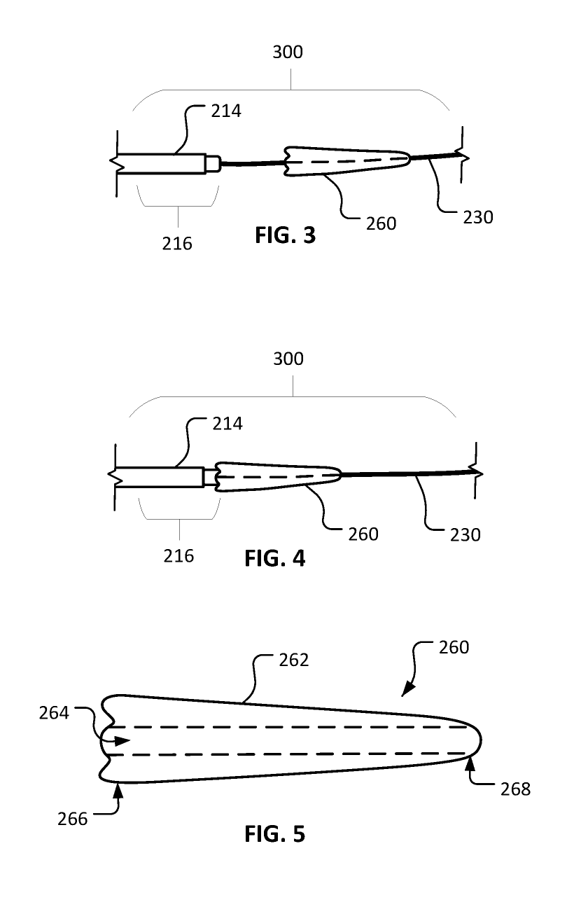

Referring to FIGS. 3 and 4, a dilation portion 300 of example integral ureteroscope and dilator system 200 includes distal end portion 216 of ureteroscope shaft 214, a portion of urologic guidewire 230, and dissolvable dilator 260. In FIG. 3, dissolvable dilator 260 is shown separated from distal end portion 216 of ureteroscope shaft 214 so that dissolvable dilator 260 is clearly distinguishable therefrom. In FIG. 4, dissolvable dilator 260 is in an operative position in relation to distal end portion 216 of ureteroscope shaft 214.

In some embodiments, dissolvable dilator 260 has a clearance fit relationship with urologic guidewire 230. In some embodiments, dissolvable dilator 260 has an interference fit relationship with urologic guidewire 230.

Referring to FIG. 5, in some embodiments dissolvable dilator 260 defines a lumen 264 that extends therethrough, between a proximal end 266 and a distal end 268 of dissolvable dilator 260. In some embodiments, dissolvable dilator 260 has a generally tapered or flared outer profile 262. That is, dissolvable dilator 260 has a generally tapered or flared outer profile 262 such that distal end 268 has a smaller outer diameter than proximal end 266. In some embodiments, dissolvable dilator 260 is generally frustoconical. In some embodiments, dissolvable dilator 260 is pseudopyramidal or otherwise graduated in diameter.

In some embodiments, distal end 268 is configured to be generally atraumatic. For example, in some embodiments distal end 268 is configured with a blunt, tapered, flared, or bullet-nose-type atraumatic tip.

In some embodiments, dissolvable dilator 260 is comprised of a material that is safely (biocompatible) and relatively quickly dissolvable within the body a patient. In some embodiments, dissolvable dilator 260 is comprised of a carbohydrate-based material. In particular embodiments, dissolvable dilator 260 is comprised of a sugar-based material. In some embodiments, dissolvable dilator 260 is comprised of a non-carbohydrate polymer suitable for the purpose. In some embodiments, dissolvable dilator 260 is comprised of multiple materials allowing for differential dissolution of various portions of dissolvable dilator 260. In some embodiments, dissolvable dilator 260 is comprised of a colloid that is suitable for the purpose.

In some implementations, dissolvable dilator 260 will essentially dissolve within a patient in a matter of minutes. For example, in some embodiments dissolvable dilator 260 will essentially dissolve in about 1 minute, about 2 minutes, about 3 minutes, about 4 minutes, about 5 minutes, about 6 minutes, about 7 minutes, about 8 minutes, about 9 minutes, about 10 minutes, or greater than 10 minutes. In some embodiments, dissolvable dilator 260 will essentially dissolve within a time range from about 30 seconds to about 1.5 minutes, within a time range from about 1 minute to about 3 minutes, within a time range from about 2 minutes to about 4 minutes, within a time range from about 3 minutes to about 5 minutes, within a time range from about 4 minutes to about 6 minutes, within a time range from about 5 minutes to about 10 minutes, or within a time range greater than 10 minutes. In some embodiments, dissolvable dilator 260 will essentially dissolve in less than 1 minute, less than 2 minutes, less than 3 minutes, less than 4 minutes, less than 5 minutes, less than 6 minutes, less than 7 minutes, less than 8 minutes, less than 9 minutes, less than 10 minutes, or greater than 10 minutes.

Because dissolvable dilator 260 dissolves in vivo after performing its dilation function, ureteroscope and dilator system 200 can be used to proceed with the treatment procedure without needing to be withdrawn (fully or partially) and without obstruction from dissolvable dilator 260. Hence, in comparison to conventional techniques, ureteroscope and dilator system 200 provides a safer, faster, less costly, and more effective technique for performing treatment procedures such as, but not limited to, ureteroscopy.

In some embodiments, dissolvable dilator 260 is obtainable as a sterile element. As such, dissolvable dilator 260 may be packaged in sterile packaging.

Dissolvable dilator 260 is scalable in size such that it can be made available in a wide variety of various sizes and configurations (e.g., taper angles, tip designs, etc.) that are suitable for the intended procedure.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A dilator device comprising:
a member defining a tapered outer profile such that a first outer diameter of a first end portion of the member is smaller than a second outer diameter of a second end portion of the member, the member defining an inner lumen extending between the first end portion and the second end portion, the lumen configured to receive a guidewire therein,
wherein the member is comprised of a material that is dissolvable within a human body without incurring harm to the human body.

2. The dilator device of claim 1, wherein the tapered outer profile comprises a frustoconical, pseudopyramidal or otherwise tapered geometric shape.

3. The dilator device of claim 1, wherein the first end portion comprises a blunt or tapered end that is configured to be atraumatic to the human body.

4. The dilator device of claim 1, wherein the material essential dissolves within the human body after being within the human body less than 10 minutes.

5. The dilator device of claim 1, wherein the material comprises a sugar-based material.

6. A dilator system comprising:
a guidewire; and
dilator device, the dilator device comprising:
a member defining a tapered outer profile such that a first outer diameter of a first end portion of the member is smaller than a second outer diameter of a second end portion of the member, the member defining an inner lumen extending between the first end portion and the second end portion, the lumen configured to receive the guidewire therein,
wherein the member is comprised of a material that is dissolvable within a human body without incurring harm to the human body.

7. The system of claim 6, further comprising an endoscope.

8. The system of claim 6, wherein the guidewire is a urologic guidewire.

9. The system of claim 8, further comprising a ureteroscope.

10. The system of claim 6, wherein the material essential dissolves within the human body after being within the human body less than 10 minutes.

11. The system of claim 6, wherein the material comprises a sugar-based material.

12. A method of performing a ureteroscopy procedure to a human patient, the method comprising:
advancing a urologic guidewire into the patient such that at least a distal end portion of the guidewire is positioned within a ureter of the patient;
advancing, over the urologic guidewire, a dilator device, the dilator device comprising:
a member defining a tapered outer profile such that a distal outer diameter of a distal end portion of the member is smaller than a proximal outer diameter of a proximal end portion of the member, the member defining an inner lumen extending between the proximal end portion and the distal end portion, the lumen receiving the guidewire therein,
wherein the member is comprised of a material that is dissolvable within the patient without incurring harm to the patient;
advancing, over the urologic guidewire and proximal of the dilator device, a ureteroscope;
dilating, by the dilator device, an orifice of the ureter;
after the dilating, allowing the dilator device to dissolve in the patient; and
after the dilating, advancing a distal end portion of the ureteroscope into the ureter.

13. The method of claim 12, wherein the dilator device dissolves in less than 10 minutes in the patient.

14. The method of claim 12, wherein the material comprises a sugar-based material.

15. The method of claim 12, further comprising capturing, using the ureteroscope, at least a portion of a kidney stone from within the ureter.
